(19) United States
(12) Patent Application Publication
(10) Pub. No.: US 2010/0106063 A1
(43) Pub. Date: Apr. 29, 2010

(54) Ultrasound Enhancing Target for Treating Subcutaneous Tissue

(75) Inventors: James E. Chomas, San Carlos, CA (US); Erin E. Girard-Hughes, Redwood City, CA (US); Sowmya Gowri Ballakur, Union City, CA (US); Doug S. Sutton, Pacifica, CA (US)

Correspondence Address: FULWIDER PATTON LLP, 6060 CENTER DRIVE, TENTH FLOOR, LOS ANGELES, CA, 90045, US

(73) Assignee: Cabochon Aesthetics, Inc., Menlo Park, CA (US)

(21) Appl. No.: 12/260,359

(22) Filed: Oct. 29, 2008

(51) Int. Cl. A61N 7/02

(52) U.S. Cl. 601/3

(57) ABSTRACT

A system for non-invasively creating a surgical lesion in subcutaneous tissue including an ultrasound generator, an ultrasound transducer operably connected to the ultrasound generator; an ultrasound enhancing target including at least two ultrasound reflecting filaments held in a spaced-apart relationship; said ultrasound generator supplying ultrasound at a pressure and frequency which will heat subcutaneous tissue and create a surgical lesion proximate the ultrasound enhancing target and will not create thermally mediated necrosis in the absence of the ultrasound enhancing target.

### FIELD OF THE INVENTION

The present invention relates to a method and apparatus for treating subcutaneous tissue using ultrasound in conjunction with an ultrasound enhancing target. The combination of ultrasound with the target of the present invention produces a localized treatment zone without concern for damaging tissue outside of the treatment zone.

### BACKGROUND

There has been a long felt need to provide a safe and effective way to harness ultrasound energy for the minimally invasive treatment of subcutaneous tissue. The disclosed invention addresses this unmet need with a system and method which enables the user to create a surgical lesion at a desired location by positioning an ultrasound enhancing target in a desired location and insonating the target with ultrasound. The target enhances the effects of ultrasound in a highly localized manner. Patient safety is assured by selecting the pressure and frequency of the ultrasound at a level which will not cause irreversible bioeffects in the absence of the enhancing target.

### SUMMARY OF THE INVENTION

Disclosed are a system and minimally invasive method for creating a surgical lesion in subcutaneous tissue. The method includes a step of providing at least one ultrasound enhancing target having at least two ultrasound reflecting filaments held in a spaced-apart relationship. The at least one ultrasound enhancing target is inserted into the subcutaneous tissue and insonated with ultrasound at a pressure and frequency below the cavitation threshold of tissue and at a duty cycle, pressure and frequency below the threshold at which tissue will undergo thermal ablation in the absence of the ultrasound enhancing target for a time sufficient to create a surgical lesion.

The above-described method may utilize ultrasound having a pressure is between 20 kPa and 10 MPa and frequency is between 200 kHz and 15 MHz.

In the above-described method the ultrasound enhancing target may be percutaneously inserted into tissue. The ultrasound enhancing target may, for example, be inserted at a depth of between 1 and 100 millimeters below the surface of the skin.

The ultrasound enhancing target may include two or more ultrasound reflecting filaments. The ultrasound reflecting filaments may have a gauge between 0.1 mm and 2 mm. The spacing between two adjacent ultrasound reflecting filaments may be between 0.1 mm and 5 mm. The spacing between two adjacent filaments is greater than zero and less than the wavelength of the ultrasound.

The ultrasound enhancing target includes a spacer sandwiched between adjacent ultrasound reflecting filaments. The filaments may be adhered or affixed to the spacer, and the spacer itself may comprise or consist of glue or epoxy.

The ultrasound enhancing target may include a first spacer sandwiched between adjacent ultrasound reflecting filaments and a second spacer which surrounds adjacent filaments.

The ultrasound reflecting filaments may be comprised of metal.

The method and system may include an active cooling element for cooling the ultrasound transducer or skin of the patient which is placed in contact with the transducer

Also disclosed is a method for creating a minimally invasive surgical lesion in subcutaneous tissue including the steps of providing at least one ultrasound enhancing target having at least two ultrasound reflecting filaments held in a spaced-apart relationship, inserting the ultrasound enhancing target into the subcutaneous tissue, insonating the ultrasound enhancing target with ultrasound at a pressure and frequency below the cavitation threshold of tissue and at a duty cycle below the threshold at which tissue will undergo thermal ablation in the absence of the ultrasound enhancing target for a time sufficient to create a surgical lesion.

Also disclosed is a minimally invasive method for creating a surgical lesion in subcutaneous tissue which includes the steps of inserting at least two ultrasound reflecting filaments into the subcutaneous tissue with a spacing of between 0.1 mm and 5 mm between adjacent filaments; insonating the at least two ultrasound reflecting filaments with ultrasound at a pressure and frequency below the cavitation threshold of tissue and at a duty cycle, pressure and frequency below the threshold at which tissue will undergo thermal mediated necrosis in the absence of the ultrasound enhancing target for a time sufficient to create a surgical lesion.

Disclosed is a system for non-invasively creating a surgical lesion in subcutaneous tissue. The system includes an ultrasound generator, an ultrasound transducer operably connected to the ultrasound generator, and an ultrasound enhancing target including at least two ultrasound reflecting filaments in a spaced-apart relationship. The ultrasound generator supplying ultrasound at a pressure and frequency which will heat subcutaneous tissue and create a surgical lesion proximate the ultrasound enhancing target and will not create thermal mediated necrosis in the absence of the ultrasound enhancing target. The ultrasound enhancing target may include a spacer sandwiched between adjacent ultrasound reflecting filaments. The filaments may be adhered to the spacer. The spacer may be epoxy. The ultrasound enhancing target may include a first spacer sandwiched between adjacent ultrasound reflecting filaments and a second spacer surrounding adjacent filaments. The system of claim may include means for actively cooling the ultrasound transducer or the skin of the patient which contacts the transducer.

Also disclosed is an ultrasound enhancing target which includes at least two ultrasound reflecting filaments and a spacer interposed between adjacent filaments, where the spacer maintains a spaced apart relationship between the ultrasound reflecting filaments.

### BRIEF DESCRIPTION OF THE DRAWINGS

FIGS. 1A and 1B are sectional drawings of a two-filament ultrasound enhancement target;

FIGS. 1C and 1D are sectional drawings of a multi-filament ultrasound enhancement target having three or more filaments; and

FIGS. 2A and 2B are sectional drawings of an ultrasound enhancement target using shrink tubing to maintain the spacing between filaments.

### DETAILED DESCRIPTION OF THE PREFERRED EMBODIMENTS

There has been a recent surge in interest in noninvasive or minimally invasive treatment of subcutaneous tissue. In particular, high intensity focused ultrasound (HIFU) is currently being investigated for use in lysing subcutaneous tissue such as fat cells. For example, several companies are actively investigating the use of HIFU to generate thermal mediated necrosis within the body. Ultrasound is used to heat tissue in the treatment zone until the tissue is injured or completely ablated. Thermal ablation requires precise targeting of the ultrasound since all tissue within the treatment zone including muscle, nerves and blood vessels is heated. Moreover, it is difficult to control the ultrasound to prevent collateral damage outside of the treatment zone because the pressure and intensity of the sound waves does not immediately diminish as they exit the treatment zone and may cause undesirable bioeffects well past the treatment zone.

Cavitation is another ultrasound modality under investigation. Cavitation entails the use of brief bursts of high intensity ultrasound to cause the implosion of bubbles within tissue in the treatment zone. Bioeffects in the tissue are caused by cavitation and may include intense local heating, streaming, pitting, and rupture of cells adjacent to the bubbles but the duration of these bursts is sufficiently short that the tissue is not significantly heated. Like the previous approach, this technique requires precise targeting to prevent damage to muscle, nerves, blood vessels and bone since anything in the target zone will experience the pressure burst. Again, due to the nonlinearities in the tissue, the focusing of sound waves is difficult to control and collateral damage outside of the treatment zone can occur.

The present inventors have discovered an altogether different approach that overcomes the problems associated with each of the aforementioned techniques. Notably, the ultrasound enhancing target of the present invention enhances the effects of ultrasound whereby a surgical lesion is created in a desired treatment zone without concern for collateral damage outside of the treatment zone. The apparatus and method of the present invention does not rely on the conventional “direct” heating of tissue in which highly focused ultrasound travels in a substantially linear path from the transducer to the focal zone of the transducer thereby causing the tissue to heat and ultimately liquefy (generate thermal mediated necrosis). The term “direct” refers to the linear path from the transducer through the treatment zone until the ultrasound exits the patient. In other words, direct heating does not intentionally utilize reflection and absorption of ultrasound within the focal zone. This treatment modality relies heavily on the ability to tightly focus the ultrasound to localize the heating to the treatment zone. Any reflection of the sound waves is incidental and unwanted since it may result in a secondary focal point and contribute to collateral damage.

The inventors have discovered a novel technique that enables the user to deliver targeted heating without the use of a sophisticated targeting system. Importantly, the present invention does not suffer from the problem of collateral damage because the ultrasound parameters are selected such that heating and the resultant surgical lesion will not occur in the absence of the ultrasound target of the present invention. Ultrasound that misses the target or propagates beyond the target will not damage the tissue because the intensity/pressure is intentionally selected such that even in the focal zone it is not sufficiently intense to cavitate or heat the tissue to induce clinically significant results (thermally mediated necrosis). Since by definition the pressure and intensity is highest in the focal zone, the pressure and intensity of any secondary focal zone will necessarily be lower.

The target of the present invention results in localized heating only in the area immediately adjacent the target.

According to a first embodiment of the invention the ultrasound enhancing target includes two or more ultrasound reflecting filaments (or filament segments) which are maintained in a spaced apart relationship from one another. It should be understood that reference herein to “filaments” (e.g., two or more ultrasound reflecting filaments) includes reference to segments of a continuously formed filament which are oriented in a spaced-apart facing relationship e.g. a coil. A target including two ultrasound reflecting filaments (or filament segments) is referred herein as a bifilar enhancing target whereas a target which includes more than two filaments (or filament segments) is known as a multifilar enhancing target. In this embodiment, uniform spacing is maintained between the filaments (or filament segments) over a treatment length where the treatment length is the length of the desired surgical lesion. However, other embodiments may incorporate variations in length, or in the uniformity of spacing between the filaments or in relation to other spacings that make up the enhancing target, without departing from the scope of the invention.

The filaments may be formed by any ultrasound reflecting material such as are commonly known in the art. Metals, such as stainless steel, copper, titanium, and any other metal that can be coated or uncoated and is of low cytotoxicity to tissue may be used. Other materials that constitutively have substantially different acoustic impedance compared to tissue, including plastics, metals, gas-filled tubes, crystals, etc. According to a present embodiment, the filaments are comprised of metal such as surgical grade stainless steel.

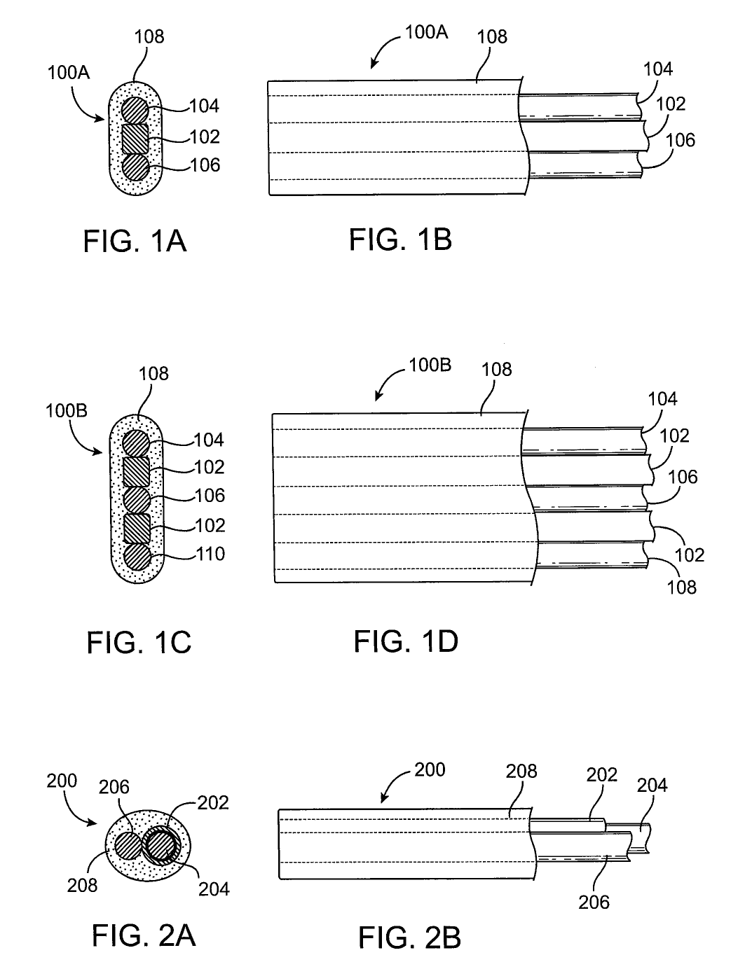

FIGS. 1A and 1B are sectional drawings of a bifilar (two filament) target 100A according to the invention. A first spacer layer 102 is sandwiched between two ultrasound reflecting filaments 104, 106. The purpose of the spacer 102 is to define the minimum spacing between the two filaments 104, 106. The filaments 104, 106 may be glued or otherwise affixed to the spacer 102. Alternatively, the filaments 104, 106 may be dipped in glue or epoxy (not shown) which will serve as spacer 102.

An optional second spacer layer 108 may surround the filaments 104, 106 and hold the filaments together. The spacer layer 108 may be formed of the same or different material from spacer layer 102. The spacer layers 102, 108 should be formed of a material which is generally transparent to ultrasound. In other words, the spacer layers should be formed of a material which is significantly less ultrasound reflective than the filaments. By manner of example, the inventors have successfully utilized polyethylene heat shrink tubing for the spacer layers 102, 108. However, any generally ultrasound transparent (non-reflecting) material may be used to maintain the spacing between the filaments.

As noted above, the purpose of the spacer layer 102 (and optional second spacer layer 108) is to control the spacing between the filaments. Thus, the spacer layers may be eliminated if one were able to insert the filaments into the treatment zone while maintaining precise control over the spacing between the filaments.

The afore-described embodiment of the ultrasound enhancing target of the invention is described in relation to a bifilar target because the invention requires at least two filament segment. Further embodiments are not limited to two filaments and can be understood to include three or more filaments (multifilar) with either equal or varied spacing between the filaments. In other embodiments, a single continuous filament which is formed so as to have two or more facing or opposing portions would essentially function the same as two or more distinct filaments.

By manner of example, FIGS. 1C and 1D is a cross-sectional drawing of a trifilar (three filament) target 100B comprising filaments 104, 106, 110 and two spacer layers 102. One spacer layer 102 is interposed between two adjacent filaments, the middle filament being disposed between each respective spacer layer.

FIGS. 2A and 2B are sectional drawings of an ultrasound enhancement target using shrink tubing to maintain the spacing between filaments. A bifilar ultrasound enhancing target 200 is shown in which filament 204 is enclosed within a first heat shrink tube 202. A second filament 206 is held in abutment with heat shrink tube 202 by a second heat shrink tube 208 which encloses both filaments 204, 206 with at least a portion of the first heat shrink tube 202 interposed therebetween.

The filaments used in a device according to the present invention are formed of an ultrasound reflecting material such as copper, stainless steel or the like. The inventors have conducted bench tests with a variety of different filament gauges and obtained satisfactory results, i.e., induced thermal medicated necrosis, using filaments between 0.1 and 2 mm. The gauges of the filaments tested were selected to minimize the gauge of the overall enhancing target and thereby maximize patient comfort. In this embodiment, the filament is between 0.1 and 2 mm. However, a smaller or larger gauge filament may be used without sacrificing efficacy.

The inventors believe that the spacing between adjacent filaments in the ultrasound enhancing target is related to the frequency of the ultrasound waves. In this embodiment the spacing between adjacent filaments is preferably between 0.1 mm and 5 mm. The spacing must be greater than zero. According to a present embodiment, the spacing is less than the wavelength of the ultrasound used to insonate the enhancing target.

The inventors have determined that the use of a multi-filar target having two or more ultrasound reflective filaments provides unexpected benefits over ultrasound with a single filament and/or ultrasound without any target. Notably, the ultrasound enhancing target of this embodiment induces a surgical lesion at a pressure/frequency below the threshold at which ultrasound alone or ultrasound with a single conductor produces any observable results. As a consequence, the risk of causing collateral damage by misdirecting the ultrasound, secondary focal zones, or propagation of ultrasound beyond the target zone is greatly diminished if not eliminated.

Devices that employ ultrasound for intense heating of tissue to create tissue damage are broadly defined by HIFU, in which a very high intensity spot is generated in the tissue. The conversion of ultrasound energy to heat is dictated by the differential impedance of the target zone compared to the surrounding tissue, and in the case of soft tissue HIFU the lesion has the same or similar impedance to that of the surrounding tissue. Therefore a mechanical lens or electronic beam forming is required to only treat the target region and spare the surrounding tissue. Very large focal gain is required to safely employ HIFU, where focal gain is the ratio of the acoustic intensity at the target region divided by the acoustic intensity at the ultrasound transducer and skin surface. The preferred parameters for a HIFU system are a center frequency between 1 MHz and 15 MHz, a large focusing aperture, acoustic intensity less than 10 W/cm2 at the skin surface, and acoustic intensities at the focus of the target region in the range of 100 W/cm2 to greater than 1000 W/cm2. These extremely high intensities can be achieved by either continuous wave ultrasound or pulsed ultrasound, and the total power delivered (i.e.—integrated acoustic pressure over time) is a key parameter. Exceptionally high acoustic intensities at the target region and short treatment time are desired to cause a very rapid thermal effect at the focus while limiting conductive heating from the target to surrounding tissue.

Devices that employ ultrasound to generate cavitation require a very large peak negative pressure at a target region in the tissue. Cavitation based systems deliver a low duty cycle burst of extremely high rarefactional (negative) pressure to elicit cavity formation in the tissue, followed by a suite of mechanisms that disrupt the cells in the treatment region. These systems utilize low frequency, in the range of 20 kHz to 2 MHz, very high mechanical index in the range of 5 to 50. In the case of cavitation-only devices, high acoustic power is not desired because it can lead to heating the tissue. Similar to the HIFU heating systems, a large aperture and high focal gain is used to achieve cavitation at the focus while sparing the surrounding tissue.

Devices which employ ultrasound to generate cavitation in structures adjacent to the ultrasound transducer, as seen in needle devices or ‘tip sonicator emulsifier’ devices generally create large and uncontrolled treatment regions around the needle tip. These devices typically use mechanical coupling of the transducer to the adjacent tissue to generate localized cavitation and emulsification of biologic tissues. They typically operate in low frequencies from 20 kHz to 500 kHz and very high mechanical indexes. They are typically defocused, meaning the peak mechanical index is adjacent to the device and then the mechanical index falls off sharply away from the device. These devices use mechanical index values in the range of 5 to 50 and necessarily are invasive in order to treat the tissue while sparing the skin and surrounding tissue.

According to one embodiment of the invention, a system is disclosed for non-invasively creating a surgical lesion in subcutaneous tissue. The system includes an ultrasound generator and an ultrasound transducer operably connected to the ultrasound generator. Also included is an ultrasound enhancing target including at least two ultrasound reflecting filaments held in a spaced-apart relationship. The ultrasound generator supplies ultrasound at a pressure and frequency which is below the cavitation threshold of tissue. Moreover, the pressure, frequency, and duty cycle of the ultrasound supplied by the generator is set below the threshold at which thermal mediated necrosis will occur in the absence of the ultrasound enhancing target. Consequently, the ultrasound will heat subcutaneous tissue and create a surgical lesion only in the tissue which is proximate the ultrasound enhancing target and will not create clinically significant heating (e.g., thermally mediated necrosis) in the absence of the ultrasound enhancing target. Clinically significant heating is heating which results in permanent, irreversible bioeffects including cell lysis. By controlling the pressure and frequency generated by the ultrasound generator, the system of this embodiment causes clinically significant heating only in the tissue proximate the enhancing target.

According to an embodiment, the ultrasound transducer device is mildly focused or planar, and the focusing is achieved by a very thin ultrasound enhancing target percutaneously placed in the tissue in a minimally invasive fashion akin to acupuncture needle placement. The ultrasound enhancing target may, for example, be inserted at a depth of between 1 and 100 millimeters below the surface of the skin. Since the ultrasound enhancing target has substantially different acoustic impedance than tissue, heating of the tissue in contact with the enhancing target can be achieved with acoustic intensities that are safe to the surrounding tissue. In this embodiment, the range of center frequencies between 200 kHz to 15 MHz is selected, and the acoustic intensity of the ultrasound is selected to be between 1 W/cm2 to 50 W/cm2 (which generally corresponds to 20 kPa to 15 MPa), with a duty cycle selected to ensure that no irreversible bioeffects occurs in tissue in the absence of the ultrasound enhancing target. In this manner, irreversible bioeffects only occur in the highly localized area proximate the ultrasound enhancing target. The term irreversible bioeffects refers to necrosis, cell lysis, thermal damage or the like.

In one embodiment, no focal gain is necessary from the ultrasound transducer, and therefore large regions containing one or many multi-filar targets can be treated. For example, a high intensity flat transducer can be used, and the transducer aperture can be large or small depending on the desired treatment region size. One advantage of a high intensity flat transducer is that it creates rapid treatment around the enhancing target and minimize conduction of heat to surrounding tissue.

Active cooling at the skin interface may be used in any of the embodiments disclosed herein to prevent or minimize surface level heating of the skin. Active cooling can be achieved by many ways, including flowing a cooling medium such as water or the like over the transducer, flowing a cooling medium (gas or liquid) through the transducer, pre-cooling the transducer, pre-cooling the tissue, or any other method to draw heat away from the skin and transducer interface. Active cooling may be utilized while the subcutaneous tissue is being insonated with ultrasound. Active cooling may be used before or after the tissue is insonated or between treatments.

According to one embodiment, the transducer is housed within a handpiece, and the handpiece includes an active cooling element in thermal communication with the transducer which cools the transducer during insonation. The active cooling element may include a cooling medium which flows over, around, or through the transducer.

Any of the methods disclosed herein may include a step of cooling the skin of the patient which will be placed contact with the transducer, and/or pre-cooling the transducer or a handpiece housing the transducer prior to treatment (insonation).

The length of the surgical lesion is directly correlated to the length of the enhancing target which is insonated. It is theorized that out-of-phase shear waves caused by the interaction of the ultrasound beam and the filaments of the enhancing target, and that the spacing between the filaments is central to the phenomenon. Only that portion of the insonated tissue which is adjacent the target is heated. Other theories exist, and the invention is not bound by any particular theory.

The inventors have observed that the lesion will generally surround and extend linearly along the length of the ultrasound enhancing target. The length of the lesion is related to the width of the beam at the location of the target. If the length of the target exceeds the width of the beam, then only that portion which is insonated will be heated. In other words, it may be necessary to sweep the transducer to insonate the full length of the enhancing target.

The inventors have determined that multiple enhancing targets may be inserted into the treatment zone to increase the overall size of the surgical lesion. The size of the lesion may also be increased by increasing the number and gauge of filaments used to form the enhancing target (two or more filaments required).

Importantly, the frequency, intensity and duty cycle of the ultrasound used to insonate the tissue is selected to avoid heating or cavitating tissue in the absence of the enhancing target. Thus, the only tissue affected by the ultrasound is that proximate the target. Consequently, the risk profile using a system according to the present invention is greatly improved over systems whose modality is cavitation or thermal ablation.

In sum, the length of the target determines the length of the surgical lesion. Thus, multiple seed-like enhancing targets may be inserted into tissue (e.g. a tumor) to be ablated. The length of the target is not important as long as the target includes at least two filaments held in a predefined spaced-apart relationship. Moreover, the target may be temporarily or permanently implanted in the patient. For example, a tumor may be seeded with numerous small seed-like ultrasound enhancing targets which may remain implanted indefinitely, thereby facilitating multiple rounds of treatment. Alternatively, the enhancing target(s) may be removed from the treatment zone after treatment is completed. It should be appreciated that the apparatus and method of the invention may be used to create a surgical lesion in any subcutaneous tissue.

According to one embodiment, two or more ultrasound reflecting filaments are inserted into the subcutaneous tissue with a spacing of between 0.1 mm and 5 mm between adjacent filaments. In other words, the tissue acts as the spacer layer. The tissue containing the filaments is insonated with ultrasound at a pressure and frequency below the cavitation threshold of tissue and at a duty cycle, pressure and frequency below the threshold at which tissue will undergo thermal mediated necrosis in the absence of the ultrasound enhancing target for a time sufficient to create a surgical lesion.

In each of the embodiments disclosed herein, insertion of the ultrasound enhancing target or the ultrasound reflecting filaments may be accomplished percutaneously through the skin. Alternatively, the filaments may be inserted through a surgical incision into the subcutaneous tissue.

In each of the embodiment disclosed herein, accurate placement of the ultrasound enhancing target or ultrasound reflecting filaments may be confirmed using ultrasound at a pressure, frequency, and duty cycle which will permit visualization without creating a surgical incision.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

What is claimed:

1. A method for creating a minimally invasive surgical lesion in subcutaneous tissue, comprising:
providing at least one ultrasound enhancing target having at least two ultrasound reflecting filament segments held in a spaced-apart relationship;
inserting the ultrasound enhancing target into the subcutaneous tissue;
insonating the ultrasound enhancing target with ultrasound at a pressure and frequency below the cavitation threshold of tissue and at a duty cycle below the threshold at which tissue will undergo thermal ablation in the absence of the ultrasound enhancing target for a time sufficient to create a surgical lesion.

2. The method of claim 1, wherein the ultrasound pressure is between 20 kPa and 15 MPa and the frequency is between 200 kHz and 15 MHz.

3. The method of claim 1, wherein the ultrasound enhancing target is percutaneously inserted into tissue.

4. The method of claim 3, wherein the ultrasound enhancing target is inserted at a depth of between 1 and 100 millimeters below a skin surface of a patient.

5. The method of claim 1, wherein the ultrasound enhancing target is percutaneously inserted into the subcutaneous tissue.

6. The method of claim 1, wherein the ultrasound reflecting filament segments have a gauge between 0.1 mm and 2 mm.

7. The method of claim 1, wherein the spacing between the at least two ultrasound reflecting filament segments is between 0.1 mm and 5 mm.

8. The method of claim 1, wherein the spacing between the at least two ultrasound reflecting filament segments is greater than zero and less than the wavelength of the ultrasound.

9. The method of claim 1, wherein the at least two ultrasound reflecting filament segments held in a spaced-apart relationship comprises a single continuous filament formed as to have two or more facing or opposing portions.

10. A minimally invasive method for creating a surgical lesion in subcutaneous tissue, comprising:
inserting at least two ultrasound reflecting filament segments into the subcutaneous tissue with a spacing between adjacent filament segments;
insonating the at least two ultrasound reflecting filaments with ultrasound at a pressure and frequency below the cavitation threshold of tissue and at a duty cycle, pressure and frequency below the threshold at which tissue will undergo thermal mediated necrosis in the absence of the ultrasound enhancing target for a time sufficient to create a surgical lesion.

11. The method of claim 10, wherein the spacing between the at least two ultrasound reflecting filament segments is between 0.1 mm and 5 mm.

12. The method of claim 10, wherein the spacing between the at least two ultrasound reflecting filament segments is greater than zero and less than the wavelength of the ultrasound.

13. The method of claim 10, wherein the at least two ultrasound reflecting filament segments held in a spaced-apart relationship comprises a single continuous filament formed as to have two or more facing or opposing portions.

14. A system for non-invasively creating a surgical lesion in subcutaneous tissue, comprising:
an ultrasound generator;
an ultrasound transducer operably connected to the ultrasound generator;
an ultrasound enhancing target including at least two ultrasound reflecting filament segments in a spaced-apart relationship; and
the ultrasound generator supplying ultrasound at a pressure and frequency which will heat subcutaneous tissue and create a surgical lesion proximate the ultrasound enhancing target and will not create thermally mediated necrosis in the absence of the ultrasound enhancing target.

15. The system of claim 14, where the ultrasound enhancing target includes a spacer sandwiched between adjacent ultrasound reflecting filament segments.

16. The system of claim 15, where the filament segments are adhered to the spacer.

17. The system of claim 15, where the spacer is an epoxy.

18. The system of claim 14, wherein the ultrasound enhancing target includes a first spacer sandwiched between adjacent ultrasound reflecting filament segments and a second spacer surrounding adjacent filament segments.

19. The system of claim 14, wherein the ultrasound reflecting filament segments are comprised of metal.

20. The system of claim 14, further comprising means for actively cooling the ultrasound transducer.

21. The system of claim 14, wherein the ultrasound reflecting filament segments have a gauge between 0.1 and 2 mm.

22. The system of claim 14, wherein the spacing between two adjacent ultrasound reflecting filament segments is between 0.1 mm and 5 mm.

23. The system of claim 14, wherein the at least two ultrasound reflecting filament segments comprise a single continuous filament formed as to have two or more facing portions.

24. A target for enhancing the effects of ultrasound, comprising:
at least two ultrasound reflecting filament segments; and
a spacer interposed between adjacent filament segments, the spacer maintaining a spaced apart relationship between the ultrasound reflecting filaments.

25. The target of claim 24, where adjacent filament segments are adhered to a respective spacer.

26. The target of claim 24, where the spacer is an epoxy.

27. The target of claim 24, wherein the target includes a first spacer sandwiched between adjacent ultrasound reflecting filament segments and a second spacer surrounding adjacent filament segments.

28. The target of claim 24, wherein the ultrasound reflecting filament segments are comprised of metal.

29. The target of claim 24, wherein the ultrasound reflecting filament segments have a gauge between 0.1 mm and 2 mm.

30. The target of claim 24, wherein a spacing between two adjacent ultrasound reflecting filament segments is between 0.1 mm and 5 mm.

31. The target of claim 24, wherein the at least two ultrasound reflecting filament segments comprises a single continuous filament formed as to have two or more facing portions.
